(19) United States
(12) Patent Application Publication
(10) Pub. No.: US 2017/0174635 A1
(43) Pub. Date: Jun. 22, 2017

(54) PROCESS FOR THE PREPARATION OF ENZALUTAMIDE

(71) Applicant: SUN PHARMACEUTICAL INDUSTRIES LIMITED, Mumbai, Maharashtra (IN)

(72) Inventors: Ramendra Singh RATHORE, Farrukhabad (IN); Venugopal Venkatarama DURVASULA, Vizianagaram (IN); Amit SHARMA, Panchkula (IN); Ram Chander ARYAN, New Delhi (IN)

(21) Appl. No.: 15/116,307

(22) PCT Filed: Jan. 30, 2015
(86) PCT No.: PCT/IB2015/050738

§ 371 Date: Aug. 3, 2016

(30) Foreign Application Priority Data
Feb. 13, 2014 (IN) 407/DEL/2014

(51) Int. Cl. C07D 233/86; C07C 231/12

(52) U.S. Cl. CPC C07D 233/86; C07C 231/12

(57) ABSTRACT

The present invention provides a process for the preparation of enzalutamide.

### FIELD OF THE INVENTION

The present invention provides a process for the preparation of enzalutamide.

### BACKGROUND OF THE INVENTION

Enzalutamide is chemically described as 4-{3-[4-cyano-3-(trifluoromethyl)phenyl]-5,5-dimethyl-4-oxo-2-sulfanylideneimidazolidin-1-yl}-2-fluoro-N-methylbenzamide of Formula I.

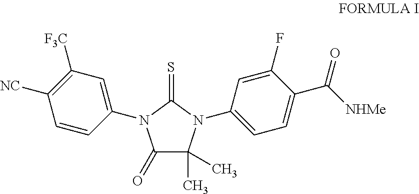 [SMILES: CNC(=O)c1ccc(N2C(=S)N(c3ccc(C#N)c(C(F)(F)F)c3)C(=O)C2(C)C)cc1F]

Processes for the preparation of enzalutamide are described in U.S. Publication Nos. 2007/0004753 and 2007/0254933; and PCT Publication Nos. WO 2007/127010, WO 2006/124118, and WO 2011/106570.

PCT Publication No. WO 2011/106570 discloses that the processes described in U.S. Publication Nos. 2007/0004753 and 2007/0254933 result in a 25% yield of enzalutamide in the final step, which accounts for a 15% overall yield. PCT Publication No. WO 2011/106570 further discloses that the known processes for preparing enzalutamide involve the use of extremely toxic reagents, for example, acetone cyanohydrin.

Therefore, there is a need in the art to develop a process for the preparation of enzalutamide that avoids the use of acetone cyanohydrin as a reagent.

### SUMMARY OF THE INVENTION

The present invention provides a process for the preparation of enzalutamide that does not involve the use of any toxic reagents and results in a higher yield of enzalutamide.

### DETAILED DESCRIPTION OF THE INVENTION

The term “about,” as used herein, refers to any value which lies within the range defined by a number up to ±10% of the value.

A first aspect of the present invention provides a process for the preparation of enzalutamide of Formula I

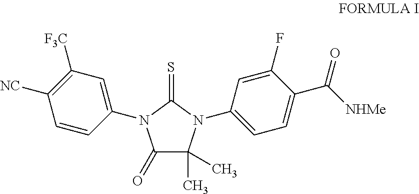 [SMILES: CNC(=O)c1ccc(N2C(=S)N(c3ccc(C#N)c(C(F)(F)F)c3)C(=O)C2(C)C)cc1F]

which comprises:
a) reacting a compound of Formula II

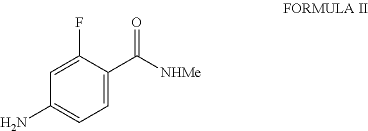 [SMILES: CNC(=O)c1ccc(N)cc1F]

with a compound of Formula III

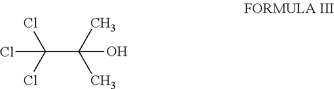

in the presence of a compound X—OH, to prepare a compound of Formula IV

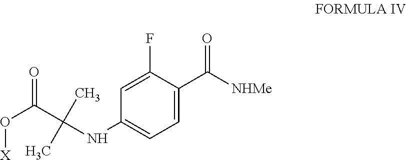

wherein X is methyl, ethyl, isopropyl, t-butyl, phenyl, or benzyl; and
b) reacting the compound of Formula IV obtained in step a) with a compound of Formula V.

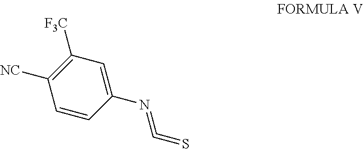 [SMILES: N#Cc1ccc(N=C=S)cc1C(F)(F)F]

A second aspect of the present invention provides a process for the preparation of a compound of Formula IV

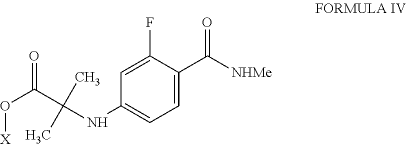

comprising reacting a compound of Formula II

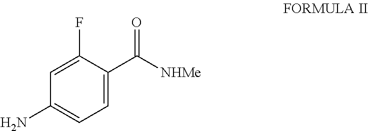 [SMILES: CNC(=O)c1ccc(N)cc1F]

with a compound of Formula III

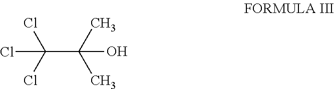

in the presence of a compound X—OH, wherein X is methyl, ethyl, isopropyl, t-butyl, phenyl, or benzyl.

The compounds of Formula II and Formula V can be prepared by any of the methods known in the art, for example, the methods disclosed in PCT Publication Nos. WO 2007/127010, WO 2006/124118 and WO 2011/106570.

In one embodiment of the present invention, the reaction of the compound of Formula II and the compound of Formula III is carried out in a solvent in the presence of a compound X—OH and optionally in the presence of a base.

The base can be an organic or an inorganic base. Examples of organic bases include ethyl amine, diisopropyl amine, diisopropyl ethyl amine, and mixtures thereof. Examples of inorganic bases include hydroxides, carbonates, and bicarbonates of an alkali or an alkaline metal, such as sodium hydroxide, potassium hydroxide, sodium carbonate, potassium carbonate, sodium bicarbonate, potassium bicarbonate, and mixtures thereof.

The solvent is selected from the group consisting of water, ethers, esters, hydrocarbons, halogenated hydrocarbons, and mixtures thereof. Examples of ether solvents include tetrahydrofuran and diisopropyl ether. Examples of ester solvents include ethyl acetate, butyl acetate, and isopropyl acetate. Examples of hydrocarbon solvents include hexane and heptane. An example of a halogenated hydrocarbon solvent is dichloromethane.

The compound of Formula X—OH is selected from the group comprising methanol, ethanol, isopropanol, t-butanol, phenol, or benzyl alcohol.

The reaction of the compound of Formula II with the compound of Formula III is carried out for about 1 hour to about 18 hours, for example, for about 1 hour to about 14 hours.

The reaction of the compound of Formula II with the compound of Formula III is carried out at a temperature of about −20° C. to about 50° C., for example, at about 0° C. to about 30° C.

The compound of Formula IV may optionally be isolated by employing one or more techniques selected from the group consisting of filtration, decantation, extraction, distillation, evaporation, chromatography, precipitation, centrifugation, concentration, and recrystallization.

In another embodiment of the present invention, the reaction of the compound of Formula IV with the compound of Formula V is carried out in a solvent. The solvent can be selected from the group consisting of water, dimethyl sulfoxide, esters, alcohols, hydrocarbons, halogenated hydrocarbons, and mixtures thereof. Examples of ester solvents include ethyl acetate, butyl acetate, and isopropyl acetate. Examples of alcohol solvents include methanol, ethanol, and n-butanol. Examples of hydrocarbon solvents include hexane and heptane. An example of a halogenated hydrocarbon solvent is dichloromethane.

The reaction of the compound of Formula IV with the compound of Formula V is carried out for about 2 hours to about 18 hours, for example, for about 4 hours to about 14 hours.

The reaction of the compound of Formula IV with the compound of Formula V is carried out at a temperature of about 10° C. to about 100° C., for example, at about 20° C. to about 95° C.

The enzalutamide compound of Formula I can be isolated by employing one or more techniques selected from the group consisting of filtration, decantation, extraction, distillation, evaporation, chromatography, precipitation, centrifugation, concentration, and recrystallization.

A third aspect of the present invention provides a process for the preparation of enzalutamide of Formula I

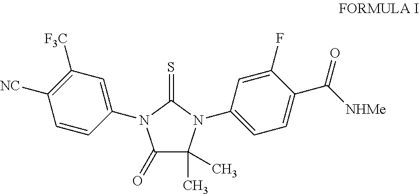 [SMILES: CNC(=O)c1ccc(N2C(=S)N(c3ccc(C#N)c(C(F)(F)F)c3)C(=O)C2(C)C)cc1F]

which comprises:
a) reacting a compound of Formula II

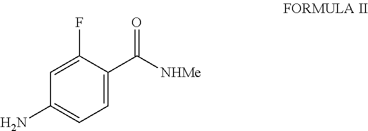 [SMILES: CNC(=O)c1ccc(N)cc1F]

with chloroform, acetone, and a compound X—OH to prepare a compound of Formula IV,

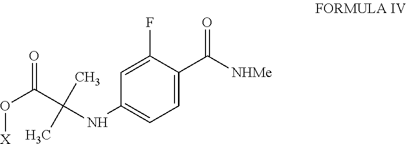

wherein X is methyl, ethyl, isopropyl, t-butyl, phenyl, or benzyl; and
b) reacting the compound of Formula IV obtained in step a) with a compound of Formula V.

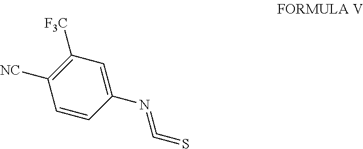 [SMILES: N#Cc1ccc(N=C=S)cc1C(F)(F)F]

A fourth aspect of the present invention provides a process for the preparation of a compound of Formula IV

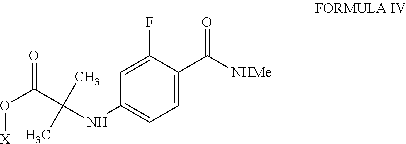

comprising reacting a compound of Formula II

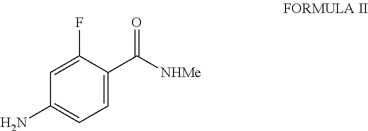 [SMILES: CNC(=O)c1ccc(N)cc1F]

with chloroform, acetone, and a compound X—OH to prepare a compound of Formula IV, wherein X is methyl, ethyl, isopropyl, t-butyl, phenyl, or benzyl group.

In an embodiment of the present invention, the compound of Formula II is reacted with chloroform, acetone, and a compound X—OH in a solvent and optionally in the presence of a base.

The base is selected from organic or inorganic bases. Examples of organic bases include ethyl amine, diisopropyl amine, diisopropyl ethyl amine, and mixtures thereof. Examples of inorganic bases include hydroxides, carbonates, and bicarbonates of an alkali or an alkaline metal, such as sodium hydroxide, potassium hydroxide, sodium carbonate, potassium carbonate, sodium bicarbonate, potassium bicarbonate, and mixtures thereof.

The solvent used for the reaction of a compound of Formula II with chloroform and acetone is selected from the group consisting of water, ethers, esters, alcohols, hydrocarbons, halogenated hydrocarbons, and mixtures thereof. Examples of ether solvents include tetrahydrofuran and diisopropyl ether. Examples of ester solvents include ethyl acetate, butyl acetate, and isopropyl acetate. Examples of hydrocarbon solvents include hexane and heptane. An example of a halogenated hydrocarbon solvent is dichloromethane.

The compound of Formula X—OH is selected from the group comprising methanol, ethanol, isopropanol, t-butanol, phenol, or benzyl alcohol.

In another embodiment of the present invention, the reaction of a compound of Formula II with chloroform and acetone is carried out in the presence of a phase transfer catalyst.

Examples of phase transfer catalysts include tetrabutylammonium iodide, tetrabutylammonium bromide, tetrabutylammonium fluoride, or mixtures thereof.

The reaction of the compound of Formula II with chloroform and acetone is carried out for about 48 hours to about 70 hours, for example, for about 48 hours to about 65 hours.

The reaction of the compound of Formula II with chloroform and acetone is carried out at a temperature of about −20° C. to about 50° C., for example, at about 0° C. to about 30° C.

The compound of Formula IV may optionally be isolated by employing one or more techniques selected from the group consisting of filtration, decantation, extraction, distillation, evaporation, chromatography, precipitation, centrifugation, concentration, and recrystallization.

The reaction of the compound of Formula IV with the compound of Formula V may be carried out as described above in earlier aspects of the present invention.

While the present invention has been described in terms of its specific embodiments, certain modifications and equivalents will be apparent to those skilled in the art and are intended to be included within the scope of the present invention.

### EXAMPLES

### Example 1

### Process for the Preparation of Ethyl N43-fluoro-4-(methylcarbamoyl)phenyl]-2-methylalaninate (Formula IV, when X=ethyl) from 1,1,1-trichloro-2-methylpropan-2-ol (Formula III)

1,1,1-Trichloro-2-methylpropan-2-ol (100 g, Formula III) was added to dichloromethane (120 mL) and the reaction mixture was cooled to 0° C. to 5° C. Sodium hydroxide (50 g) was added to the reaction mixture and the mixture was stirred for 30 minutes. N-Methyl 2-flouro-4-amino benzamide (10 g) and ethanol (30 mL) were added to the reaction mixture at 0° C. to 5° C. over 1 minute. The reaction mixture was stirred at 0° C. to 5° C. for 60 minutes. The reaction mixture was heated at 20° C. to 25° C. for 2 hours to 3 hours. Water (100 mL) and dichloromethane (100 mL) were added to the reaction mixture and the mixture was stirred for 15 minutes. The layers obtained were separated, and then the organic layer was concentrated at 45° C. to 50° C. over 1 hour to 2 hours to obtain the title compound.

Yield: 12 g.

### Example 2

### Process for the Preparation of Ethyl N43-fluoro-4-(methylcarbamoyl)phenyl]-2-methylalaninate (Formula IV, when X=ethyl)

N-Methyl 2-flouro-4-amino benzamide (0.3 g), chloroform (0.3 mL), acetone (2 mL), and tetrabutylammonium iodide (0.001 g) were added to dichloromethane (4 mL) and ethanol (0.4 mL). The reaction mixture was cooled to 0° C. to 5° C. and a solution of sodium hydroxide (0.36 g) in water (0.7 mL) was added to the reaction mixture. The reaction mixture was stirred at 0° C. to 5° C. for 48 hours. A mixture of water (10 mL) and dichloromethane (10 mL) was added to the reaction mixture and the mixture was stirred for 15 minutes. The layers obtained were separated, and then the organic layer was concentrated to obtain the residue. The residue obtained was purified using a silica gel column to obtain the title compound.
Yield: 0.15 g.

### Example 3

### Process for the Preparation of Enzalutamide (Formula I)

Ethyl N-[3 -fluoro-4-(methylcarbamoyl)-phenyl]-2-methylalaninate (0.2 g, Formula IV, when X=ethyl) and 4-isothiocyanato-2-(triflouromethyl)-benzonitrile (0.33 g, Formula V) were added to dimethyl sulfoxide (0.2 mL) and isopropyl acetate (0.4 mL) and the mixture was heated to 90° C. to 95° C. The reaction mixture was cooled to 70° C., followed by the addition of methanol (0.4 mL). The reaction mixture was stirred for 2 hours. Isopropyl acetate (4 mL) was added to the reaction mixture, and the mixture was washed with water (4 mL). The layers obtained were separated, and the organic layer was concentrated at 35° C. under vacuum to obtain an oily residue. The oily residue obtained was purified using a silica gel column to obtain the title compound.
Yield: 0.2 g

1.-15. (canceled)

16. A process for the preparation of enzalutamide of Formula I 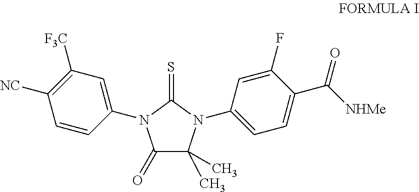 [SMILES: CNC(=O)c1ccc(N2C(=S)N(c3ccc(C#N)c(C(F)(F)F)c3)C(=O)C2(C)C)cc1F]
which comprises:
a) reacting a compound of Formula II 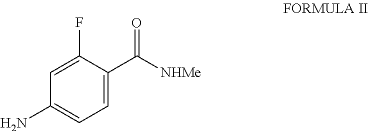 [SMILES: CNC(=O)c1ccc(N)cc1F]
with a compound of Formula III 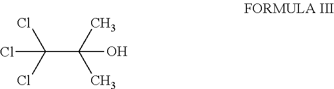
in the presence of a compound X—OH, to prepare a compound of Formula IV 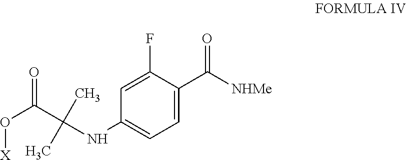
wherein X is methyl, ethyl, isopropyl, t-butyl, phenyl, or benzyl; and
b) reacting the compound of Formula IV obtained in step a) with a compound of Formula V.

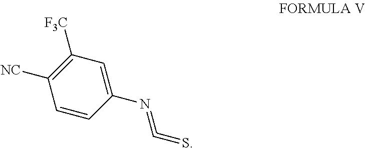 [SMILES: N#Cc1ccc(N=C=S)cc1C(F)(F)F]

17. The process according to claim 16, wherein the reaction of the compound of Formula II with the compound of Formula III is carried out in a solvent.

18. The process according to claim 17, wherein the solvent is selected from the group consisting of water, ethers, esters, hydrocarbons, halogenated hydrocarbons, and mixtures thereof.

19. The process according to claim 16, wherein the reaction of the compound of Formula II with the compound of Formula III is carried out in the presence of a base.

20. The process according to claim 16, wherein the reaction of the compound of Formula IV with the compound of Formula V is carried out in a solvent.

21. A process for the preparation of a compound of Formula IV 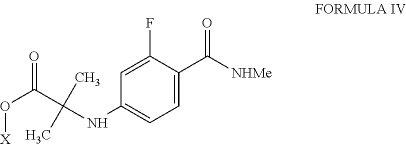
wherein X is methyl, ethyl, isopropyl, t-butyl, phenyl, or benzyl group comprising reacting a compound of Formula II 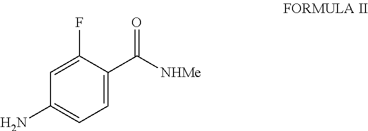 [SMILES: CNC(=O)c1ccc(N)cc1F]
with a compound of Formula III 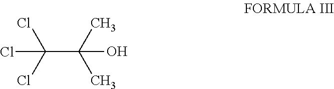
in the presence of X—OH, wherein X is methyl, ethyl, isopropyl, t-butyl, phenyl, or benzyl.

22. The process according to claim 21, wherein the reaction of the compound of Formula II with the compound of Formula III is carried out in a solvent.

23. The process according to claim 22, wherein the solvent is selected from the group consisting of water, ethers, esters, hydrocarbons, halogenated hydrocarbons, and mixtures thereof.

24. The process according to claim 21, wherein the reaction of the compound of Formula II with the compound of Formula III is carried out in the presence of a base.

25. A process for the preparation of enzalutamide of Formula I 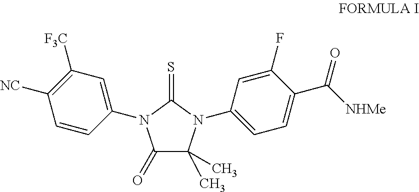 [SMILES: CNC(=O)c1ccc(N2C(=S)N(c3ccc(C#N)c(C(F)(F)F)c3)C(=O)C2(C)C)cc1F]
which comprises:
a) reacting a compound of Formula II 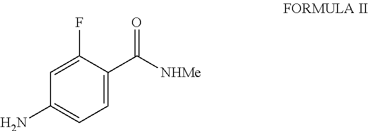 [SMILES: CNC(=O)c1ccc(N)cc1F]
with chloroform, acetone, and X—OH to prepare a compound of Formula IV 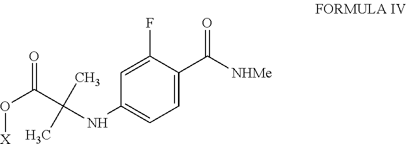
wherein X is methyl, ethyl, isopropyl, t-butyl, phenyl or benzyl; and
b) reacting the compound of Formula IV obtained in step a) with a compound of Formula V.

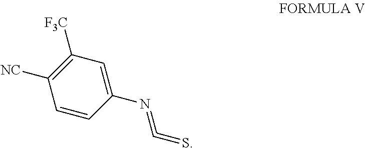 [SMILES: N#Cc1ccc(N=C=S)cc1C(F)(F)F]

26. The process according to claim 25, wherein the compound of Formula II is reacted with chloroform, acetone, and the compound X—OH in a solvent.

27. The process according to claim 26, wherein the solvent is selected from the group consisting of water, ethers, esters, alcohols, hydrocarbons, halogenated hydrocarbons, and mixtures thereof.

28. The process according to claim 25, wherein the compound of Formula II is reacted with chloroform, acetone, and the compound X—OH in the presence of a base.

29. The process according to claim 25, wherein the reaction of the compound of Formula II with chloroform and acetone is carried out in the presence of a phase transfer catalyst.

30. The process according to claim 25, wherein the reaction of the compound of Formula IV with the compound of Formula V is carried out in a solvent.

31. A process for the preparation of a compound of Formula IV 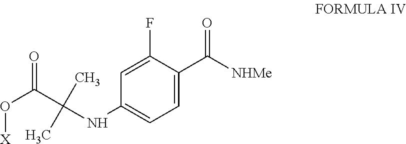
wherein X is methyl, ethyl, isopropyl, t-butyl, phenyl or benzyl comprising reacting a compound of Formula II 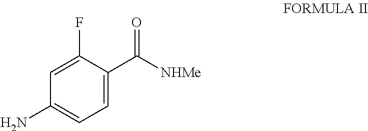 [SMILES: CNC(=O)c1ccc(N)cc1F]
with chloroform, acetone, and a compound X—OH to prepare the compound of Formula IV, wherein X is methyl, ethyl, isopropyl, t-butyl, phenyl, or benzyl.

32. The process according to claim 31, wherein the compound of Formula II is reacted with chloroform, acetone, and the compound X—OH in a solvent.

33. The process according to claim 32, wherein the solvent is selected from the group consisting of water, ethers, esters, alcohols, hydrocarbons, halogenated hydrocarbons, and mixtures thereof.

34. The process according to claim 31, wherein the compound of Formula II is reacted with chloroform, acetone, and the compound X—OH in the presence of a base.

35. The process according to claim 31, wherein the reaction of the compound of Formula II with chloroform and acetone is carried out in the presence of a phase transfer catalyst.
